(19) United States
(12) Patent Application Publication
(10) Pub. No.: US 2019/0333496 A1
(43) Pub. Date: Oct. 31, 2019

(54) SPATIALIZED VERBALIZATION OF VISUAL SCENES

(71) Applicant: YISSUM RESEARCH DEVELOPMENT COMPANY OF THE HEBREW UNIVERSITY OF JERUSALEM LTD., Jerusalem (IL)

(72) Inventors: Amir AMEDI, Jerusalem (IL); Tomer BEHOR, Rishon Lezion (IL)

(21) Appl. No.: 16/349,950

(22) PCT Filed: Nov. 14, 2017
(86) PCT No.: PCT/IL2017/051237

§ 371 Date: May 14, 2019

Related U.S. Application Data
(60) Provisional application No. 62/421,472, filed on Nov. 14, 2016.

(51) Int. Cl. G10L 13/027; G09B 21/00; A61F 9/08

(52) U.S. Cl. CPC G10L 13/027; A61F 9/08; G09B 21/007

(57) ABSTRACT

A system that comprises at least one hardware processor, which is configured to: receive a digital image of a scene; analyze the image to identify one or more objects appearing in the image; and for each identified object: (i) determine values for a plurality of physical attributes of the respective identified object, (ii) synthesize a vocalized verbal description of the respective identified object, wherein at least some of the values of said plurality of physical attributes are expressed by non-verbal audio parameters of the synthesized vocalized verbal description, and (iii) output said synthesized vocalized verbal description through a loudspeaker or an earphone.

### CROSS-REFERENCE TO RELATED APPLICATIONS

This application claims priority to U.S. Provisional Patent Application No. 62/421,472, filed Nov. 14, 2016, entitled “The TopSpeech Algorithm: A Novel Topographic Approach for Conveying Multiple Objects in a Scene through Spatialized Verbalization”, which is incorporated herein by reference in its entirety.

### BACKGROUND

The invention relates to the field of sensory substitution systems and auditory representation of visual scenes.

Sensory substitution and/or verbalization systems and devices are human-machine interfaces which receive information via one modality (for example, visually) and translate it into another modality (for example, auditory or haptic), through which the information is then perceived by a user. In one example, such a system may receive as input visual information of the physical world via, e.g., a camera, substitute it into auditory cues via a pre-determined algorithm, and then convey the auditory information to a user via headphones, bone-conductors, or other means, so as to enable the user access to the visual information through the auditory modality.

Deep learning algorithms are already reaching an impressive level of accuracy and speed, with a growing list of daily-life applications. However, a key challenge arising from these advances is how to efficiently convey the complex output of these algorithms to the human brain in real-life situations, without creating a cognitive overload or reducing the amount of information coming from the visual modality. Some solutions focus mainly on the auditory modality, by sonically representing the visual elements in a scene by abstract sounds, or by an exhaustive verbal description of the image content. However, these approaches are inefficient in describing complex scenes, and are limited in their ability to provide users with accurate, quickly-acquired knowledge of the position and identities of elements in the scene.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with certain embodiments of the present disclosure, a system comprising at least one hardware processor configured to: receive a digital image of a scene; analyze the image to identify one or more objects appearing in the image; and, for each identified object (i) determine values for a plurality of physical attributes of the respective identified object, (ii) synthesize a vocalized verbal description of the respective identified object, wherein at least some of the values of said plurality of physical attributes are expressed by non-verbal audio parameters of the synthesized vocalized verbal description, and (iii) output said synthesized vocalized verbal description through a loudspeaker or an earphone.

There is also provided, in accordance with certain embodiments of the present disclosure, a method comprising using at least one hardware processor for receiving a digital image of a scene; analyzing the image to identify one or more objects appearing in the image; and for each identified object (i) determining values for a plurality of physical attributes of the respective identified object, (ii) synthesizing a vocalized verbal description of the respective identified object, wherein at least some of the values of said plurality of physical attributes are expressed by non-verbal audio parameters of the synthesized vocalized verbal description, and (iii) outputting said synthesized vocalized verbal description through a loudspeaker or an earphone.

There is further provided, in accordance with certain embodiments of the present disclosure, a computer program product, the computer program product comprising a non-transitory computer-readable storage medium having program code embodied therewith, the program code executable by at least one hardware processor to receive a digital image of a scene; analyze the image to identify one or more objects appearing in the image; and, for each identified object (i) determine values for a plurality of physical attributes of the respective identified object, (ii) synthesize a vocalized verbal description of the respective identified object, wherein at least some of the values of said plurality of physical attributes are expressed by non-verbal audio parameters of the synthesized vocalized verbal description, and (iii) output said synthesized vocalized verbal description through a loudspeaker or an earphone.

In some embodiments, the plurality of non-verbal audio parameters are selected from the group consisting of: pitch, volume, timbre, speed, voice gender, number of voices used, type of voice used, language, accent, emotion expressed by the voice, echo, and reverberation.

In some embodiments, the plurality of physical attributes are selected from the group consisting of: location in the horizontal dimension, location in the vertical dimension, location in the depth dimension, height, width, size, color, depth, weight, texture, and temperature.

In some embodiments, the object is a human, wherein said plurality of physical attributes are selected from the group consisting of: identity, sex, height, weight, age, nationality, and emotional state or mood. In some embodiments, the object comprises at least one of textual information and symbolic information, and wherein said identification comprises detection of said textual or symbolic information. In some embodiments, the object comprises a virtual representation of a physical object.

In some embodiments, the identification comprises retrieving information with respect to said identified object from at least one of a database of the system, an external network resource, a cloud server, and the Internet.

In some embodiments, a plurality of said synthesized vocalized verbal descriptions corresponding to a plurality of objects disposed in different locations about a said image, are combined into a continuous sequence in a specified order, based on the relative locations of said plurality of objects in the image. In some embodiments, the specified order is selected from the group consisting of: left-to-right, right-to-left, top-to-bottom, and bottom-to-top.

In some embodiments, the vocalized verbal description of said respective identified object comprises two or more concurrent vocalized verbal descriptions.

In some embodiments, the at least one hardware processor is further configured to: slice the image into a plurality of slices; detect, in a specified order for each slice at a time, the location of at least a portion of the object contained within the slice; associate a sound or tactile object-dependent signal with the object; associate a sound or tactile location-dependent signal unique for each slice; combine in the specified order each object-dependent signal with a respective location-dependent signal for creating a combined object-location signal; and output the combined object-location signal concurrently with said synthesized vocalized verbal description.

In some embodiments, each of the non-verbal audio parameters is associated with a unique physical attribute, based on user selection.

In some embodiments, at least some of the determined values for said plurality of physical attributes of a said identified object are expressed by haptic signals, wherein said haptic signals are being output concurrently with said vocalized verbal description of the respective identified object. In some embodiments, a said physical attribute is distance and said haptic signal is a vibratory signal. In some embodiments, at least some of the determined values for said plurality of physical attributes of a said identified object are expressed by non-vocal audio signals, wherein said non-vocal audio signals are being output concurrently with said vocalized verbal description of the respective identified object. In some embodiments, a said physical attribute is color and said non-vocal audio signals are sounds associated with musical instruments, wherein each color is represented by a different musical instrument.

In some embodiments, the system further comprising a user interface unit comprising one or more of a microphone, bone-conducting headphones, tactile glove, haptic device, and a virtual or augmented reality engine.

In some embodiments, the image is generated by an imaging method selected from the group consisting of optical imaging, two-dimensional imaging, three-dimensional imaging, radio frequency imaging, ultrasound imaging, and infrared imaging.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A illustrates an exemplary implementation of the present system, according to some embodiments;

FIG. 1B shows a flowchart illustrating a method for conveying physical attributes of objects in a scene through non-verbal audio parameters of vocalized verbal descriptions, according to some embodiments;

FIG. 2A shows a block diagram of an exemplary embodiment of a system for conveying physical attributes of objects in a scene through non-verbal audio parameters of vocalized verbal descriptions, according to some embodiments; and

FIGS. 2B-2D illustrate the use of additional user-interface devices and implements, according to some embodiments.

### DETAILED DESCRIPTION

Disclosed herein are a system and a method for conveying properties of an environment or a scene through non-visual sensory representation, such as auditory, haptic, or other sensory representation.

The present disclosure allows for intuitive and quick perception of an entire scene containing a plurality of physical and/or virtual objects, including, among other properties, their identity or type, relative spatial positions, distance, size, color, and/or similar properties. In some embodiments, the present technology may also provide facial recognition, and/or convey the properties of textual, semantic, contextual, and/or symbolic information contained in the environment. As such, it may be of particular interest in the field of systems and methods for assisting the visually impaired. However, it will be appreciated that numerous other applications of this technology may be considered.

In the present disclosure, the term “scene” is intended to cover (i) any physical environment comprising any indoors, outdoors, cityscape, countryside, landscape, terrain, and/or similar views, (ii) any virtual environment, and (iii) any individual objects in isolation within such environments.

The term “object” refers to (i) any physical whose spatial characteristics and attributes may be detected by at least one specified imaging device, (ii) any virtual object, (iii) any person who may be identified using facial recognition, and (iv) any textual, symbolic, or semantic information contained in a scene or an environment, which may be detected by at least one specified detection device.

The term “image” refers to any image or portion of an image that includes a representation of an object or a scene.

The term “imaging device” is broadly defined as any device that captures images or other representations of objects and represents them as a digital two-dimensional or three-dimensional (3D) image. Imaging devices may be optic-based, such as image sensors, but may also include depth sensors, radio frequency imaging, ultrasound imaging, infrared imaging, and the like.

As noted above, with the advancements of computer vision algorithms, complex physical scenes, with multiple objects, can be accurately and quickly detected and recognized by computers and mobile devices using an imaging device. These developments, however, raise the challenge of how to convey this wealth of information, which is only about to increase, to a user. Conveying an algorithm's output solely through visual means suffers from several disadvantages, primarily because the visual modality is the one we already rely on the most in our daily life. Thus, adding more visual information can actually reduce the efficacy of the constant stream of input from this modality, by creating a cognitive load. Moreover, conveying visual information, by necessity, relies on presenting such information within the available field-of-view of the user. This may pose a problem where the attention of the user should be focused in other areas, such as when driving; when there is a need to augment more information on top of an already complex visual scene; or when the user has visual impairment. These reasons clearly point to the growing need for a way to efficiently create an eyes-free representation of visual information through other modalities, such as sound.

Accordingly, the present disclosure employs a “topographic speech” (TS) approach, which represents objects in a scene through vocalized verbal descriptions, while at the same time conveying physical and topographical properties of the objects with or without relation to space, such as position, size, height and/or color, through different auditory characteristics of the vocalized verbal descriptions. These auditory characteristics may include, but are not limited to, pitch, volume, speed, and/or timbre. This approach takes advantage of the inventors' discovery that humans are able to intuitively interpret manipulations in auditory properties (e.g., pitch, volume, and/or timbre) as topographic cues. A complementary method further discussed below for representing visual images by alternative senses was disclosed by the present inventors in PCT International Application No. PCT/IB2010/054975, International Filing Date Nov. 3, 2010, published on May 12, 2011 as International Patent Application Publication No. WO 2011/055309, which is incorporated herein by reference.

In some embodiments, the present system receives as an input an image of a scene, and conveys the identity and/or type of objects in the scene by outputting vocalized verbal descriptions of each such object. At the same time, the spatial locations and other physical attributes of these objects (such as their size, height, color, and/or other attributes) are conveyed through non-verbal audio parameters of the vocalized descriptions (such as the pitch, volume, timbre, and/or speed of the speech; e.g., the pitch level of the speech may convey the height of the object). In some variations, some physical attributes may be represented by outputting haptic sensory signals or non-vocal audio signals, concurrently with the vocalized descriptions.

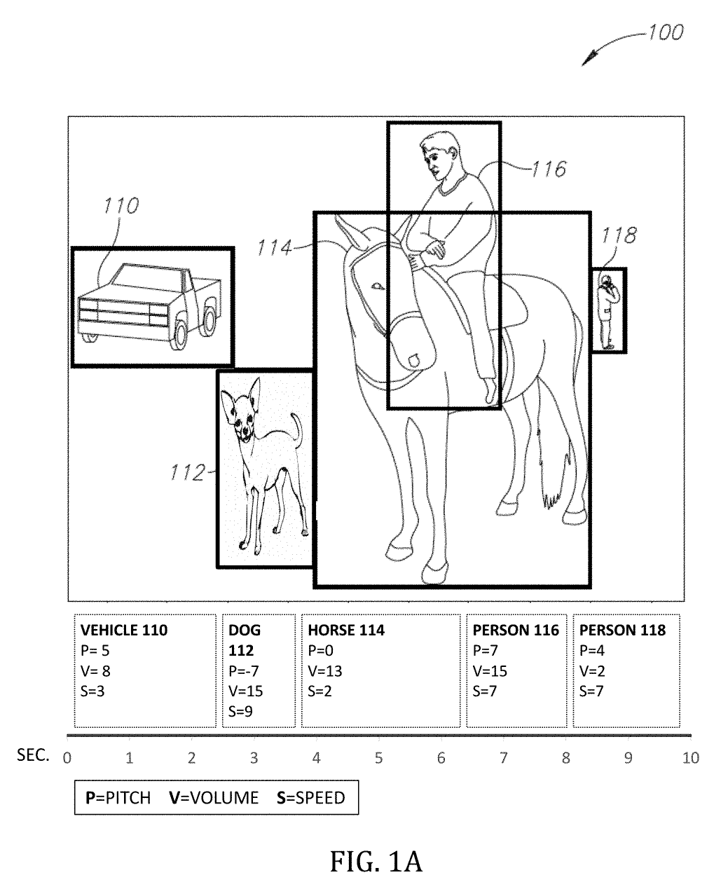

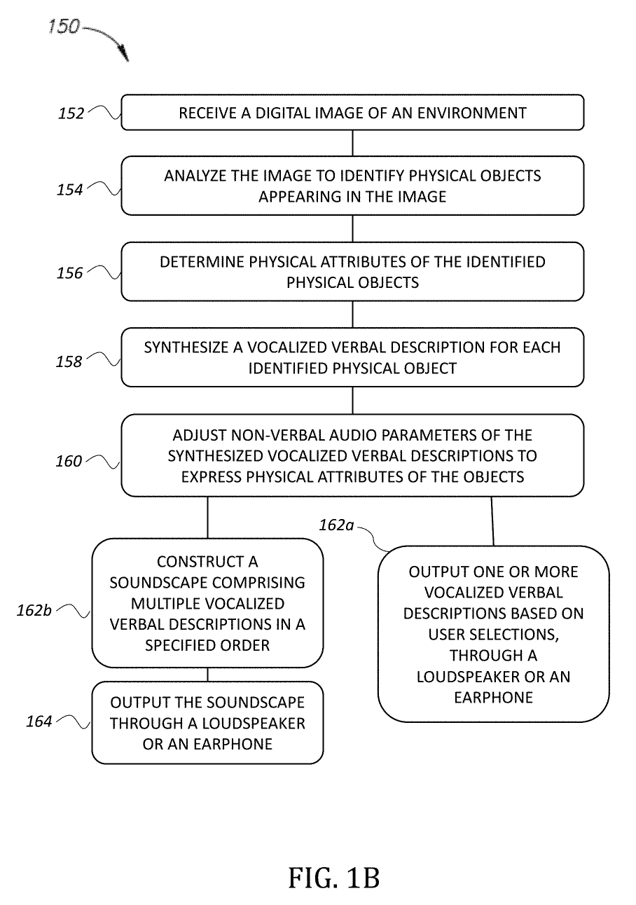

FIG. 1A illustrates an exemplary implementation of the present system, according to some embodiments. A scene 100 contains several objects, e.g., vehicle 110, dog 112, horse 114, and persons 116, 118. The scene is scanned using an imaging device, e.g., three-dimensional (3D) scanning equipment and an image is generated. The image is then analyzed using dedicated computer vision and image processing algorithms (e.g., YOLO, R-CNN; or facial recognition algorithms such as OpenFace, and FaceNet), which produce an output identifying the objects in scene 100; their relative spatial locations in the vertical, horizontal, and depth dimensions within the scene; and their various physical attributes (such as size, height, and/or color). This data is processed under a topographic speech protocol, and a vocalized verbal description of each object in scene 100 is synthesized. At least some of the physical attributes of each object in scene 100 may then be expressed by non-verbal audio parameters of such object's vocalized verbal description. The vocalized verbal descriptions for scene 100 may then be output to a user, e.g., via a loudspeaker or earphones. In some variations, individual vocalized verbal descriptions may be output one at a time, based on user selection or system configuration. For example, only objects that are in a user's path, or indicated by the user as objects of interest, may be represented by an output of the relevant vocalized verbal description(s). Alternatively, multiple vocalized verbal descriptions corresponding to some or all of the objects in scene 100 may be combined into a soundscape, i.e., a single continuous sequence of vocalized verbal descriptions, thus providing a partial or complete overview of scene 100. In some embodiments, the individual vocalized verbal descriptions comprising such soundscape are combined and output in a specified order, based, e.g., on the relative spatial locations of the various objects in scene 100. In some variations, the specified order may be left-to-right (e.g., the rightmost object will be vocalized last), right-to-left, top-to-bottom, or bottom-to-top. In other variations, the specific order may be dependent, e.g., on the type of objects in the scene, their relative size, or distance from the user.

With reference to FIG. 1A, a soundscape of scene 100 may provide a representation of scene 100 in its entirety from left to right. The time scale at the bottom of scene 100 in FIG. 1A provides an illustration of the timing and duration within the soundscape of the respective individual vocalized verbal descriptions of each of the objects in scene 100. The information provided adjacent to the time scale details the non-verbal audio parameters of the vocalized verbal descriptions used to describe each object in scene 100 (wherein “p” refers to pitch, “v” refers to volume, and “s” refers to speed). It will be appreciated that the order of introducing the several objects in a scene and the overall length of the soundscape are dependent on numerous parameters, including the number of objects, their sizes and locations, as well as, in some embodiments, user-selected parameters, or predetermined configuration of the system. It will further be appreciated that the respective timings, durations, and other values given in the following discussion are given for illustrative and exemplary purposes only, and that varying timings, durations, and values may be assigned to objects based on a plurality of parameters, predetermined configuration of the system, or user-selected settings.

Thus, for example, the soundscape of scene 100, comprising vocalized verbal descriptions of all objects in scene 100, may first identify vehicle 110 (soundscape time approximately 0-2.25 seconds as illustrated by the time scale in FIG. 1A), by vocalizing the word “vehicle” or “truck.” The vocalized verbal description may have one or more of its non-verbal audio parameters used to convey the relative spatial location of vehicle 110 in scene 100. Accordingly, the vocalized word “vehicle” may be read using a nominal pitch level 5 (e.g., on a scale of −10 to 10) to denote a vertical position of a geometric center point of vehicle 110; and using a nominal volume level of 8 (e.g., on a scale of 1 to 20) to denote the relative location in the depth dimension, or distance from the observer, of vehicle 110 in scene 100. The location and size of vehicle 110 in the horizontal dimension may be represented using the word “vehicle” being vocalized at nominal speed level 3 (e.g., on a scale of 1 to 10) to denote the horizontal width of vehicle 110. Accordingly, in some embodiments, the word “vehicle” in reference to vehicle 110 may be vocalized at a speed in which the duration of its pronunciation, approximately 2.25 seconds in the present case, is proportional to the relative horizontal footprint of the object within the scene.

Next, dog 113 may be represented by the vocalized word “dog,” with suitable non-verbal audio parameters of the vocalized description, to denote the spatial location and other physical attributes of dog 112 in scene 100. Next, Horse 114 and person 116 may be represented using relevant vocalized verbal descriptions. For example, in the case of horse 114, the vocalized verbal description (i.e., the word “horse”) may begin at approximately 3.75 seconds into the soundscape, and be read at a relatively slow speed, over, e.g., 2.75 seconds, to signify the space occupied by horse 114 in the horizontal dimension of scene 100. The vocalized verbalization for person 116 may then begin at approximately 6.5 seconds into the soundscape, and lasting for a shorter overall duration of approximately 1.5 seconds. In other words, the word “horse” in reference to horse 114 will be introduced before, and be read slower than, the word “person,” in reference to person 116, which will also be read in a higher pitch to signify its higher vertical position in the vertical dimension. The respective vocalized verbal descriptions for horse 114 and person 116 will of course each display suitable other non-verbal audio parameters, such as volume and/or other parameters, as shown in FIG. 1A, to signify other attributes of the respective objects. Thus, may the soundscape unfold until all objects have been similarly represented.

It will be appreciated that the spatial or three-dimensional location of an object in a scene may be conveyed variously based on (a) representing the location of a single physical point of an object (e.g., a geometrical center point) in the horizontal and vertical dimensions, or (b) representing the full height and width of the object, based on its horizontal and vertical boundaries. Similarly, location in the depth dimension may be represented, e.g., as the distance from the observer to the closest point of the object, or, alternatively, reflect the full extent of the object in the depth dimension. These alternative ways of representing spatial location and size of objects in the vertical, horizontal, and depth dimensions, may be used independently of one another, e.g., based on user selection. In other words, in some instances, location in the vertical dimension may be represented as a single point, while locations in the horizontal and depth dimensions may be represented as the full size of the object, etc. As an example, an object's height or location in the vertical dimension of the scene may be conveyed by a single vocalized verbal description conveying the height of a center point thereof, or alternatively by more than one concurrent vocalized verbal descriptions, each using a different pitch (where pitch represents location in the vertical dimension of a scene), based, e.g., on the top-most and bottom-most boundaries of the object. For example, with continuing reference to FIG. 1A, person 116 may be spatially represented by using a single vocalization of the word “person” with mid-high pitch (e.g., nominal level 7), to signify a center point located in the upper section of scene 100. Alternatively, person 116 may be represented by two concurrent vocalizations of the word “person,” using mid-level and high-level pitches, respectively, to signify the vertical boundaries of person 116 in scene 100.

Similarly, location and width of an object in the horizontal dimension may be represented through suitable changes to non-verbal audio parameters of the relevant vocalized verbal descriptions. For example, the width of person 116 (i.e., the total footprint in the horizontal dimension of person 116) may be represented through vocalizing the word “person” over a period of, e.g., 1.5 seconds, such that the length of the vocalization may represent the relative horizontal footprint of person 116.

It will be appreciated that the present system may be configured such that the associations of non-verbal audio parameters of vocalized descriptions (e.g., pitch, volume, speed, and/or timbre) with physical attributes (e.g., size, height, depth, and/or color) may be preconfigured, replaced with others, or exchanged between each other according to needs, or subject to modification by a user. Accordingly, for example, volume (loud or low) may variously be associated with size (large or small), distance (close or far), or color (light or dark). The same is true for any other non-verbal audio parameter/physical attribute association in the present disclosure, and any specific associations discussed herein are by way of example only and should not be interpreted as limiting in any way.

In some embodiments, non-verbal audio parameters which may be used to convey spatial information may include, but are not limited to: voice pitch, voice timbre, different genders and types of voices (e.g., male/female or adult/child voices, etc.), different accents or languages used, different emotional states or moods intended to be expressed by the voice (e.g., happiness, anger), and/or different auditory effects on the vocalized description, such as echo or reverberation. Each of these parameters may in turn be associated with one of the physical properties of an object, including color, depth, weight, texture, or temperature; as well as more specific properties related to specific objects, such as the gender of a face, the emotional state or mood expressed by a person, and/or the nationality of a person.

It will be further appreciated that the present system and method may also be used in various other applications. For example, the system may comprise facial recognition algorithms, such that the system may identify individuals by name, gender, age, other physical attributes, and/or verbal or physical interactions with their surrounding environment. For example, in some variations, the system may provide individuals' relative spatial position in a room, as well as information about other individuals and/or objects with which they interact, e.g., through speech, verbal reference, or physical contact.

The present system and method may also be used in text recognition, thus verbalizing a written text detected in the scene. The text can be rendered physically, e.g., in a road sign, a paper document, and/or a restaurant menu; or it can be displayed on a computer screen and/or other electronic display. The system may then read the text, while conveying additional attributes thereof, such as spatial position, semantic context, color, etc.

The present system and method may also be employed to represented scenes in the context of virtual or augmented reality, in which objects are virtual representations with no physical existence. In some embodiments, a virtual reality or augmented reality engine can be integrated with the present system, in areas such as gaming or for expanding sensory abilities of humans. In one example, such integration may enable users to expand their effective field-of-view using the present system, by receiving vocalized verbal descriptions (potentially in combination with the EyeMusic algorithm discussed below) regarding objects outside of the visual field-of-view (e.g., from the sides or from behind the user).

In some variations, the system may convey supplemental information about objects which may not be visually and/or physically detectable, based on data preprogrammed in the system and/or data obtained by accessing an external network resource, such as a cloud server or the Internet. For example, when an object is partly occluded, or when visibility is low, the system may supplement visually-received data with known data about the particular type of object. In another example, in the context of shopping, the system may recognize an object being considered for purchase, and describe its visually-perceived attributes. Upon a prompt by a user, the system may then access a database or the Internet, to obtain supplemental information about such object, including price, user reviews, and/or availability, etc. The system may also employ, e.g., an infrared sensor and/or other night vision means, for a more accurate detection of objects in darkness. The use of infrared or other thermal sensors may also be able to convey additional information detectable by such means, for example, temperature of an object or the ambient environment.

In some embodiments, a scene may be conveyed from a point of view other than that of the user of the system, e.g., from in front of, behind, the sides, and/or above, the user (e.g., by an imaging device operated by another person in the scene, or by imaging from a ceiling camera or a drone). In such cases, the represented scene will include the user as an object of the scene.

In another embodiment, a topographic speech algorithm, potentially in combination with the EyeMusic algorithm discussed below, can be used in a multisensory rehabilitation program. In such cases, the topographic speech representation is conveyed alternately with and without a display of the scene being so represented, as a means of training users on the system. Such training may involve controlled environments with varying numbers, types, and locations of objects, as well as ‘live’ scenes in the real world. The same training method can also apply to texts, and thus it is not limited to objects. In some cases, this training can be used, for example, with patients who suffer from degenerative eye diseases, but still retain a measure of seeing.

FIG. 1B is a flowchart of a method 150 for conveying physical attributes of objects in a scene through non-verbal audio parameters of vocalized verbal descriptions. Method 150 may include a step 152 for receiving a digital image of a scene. In a step 154, the digital image is analyzed, to identify one or more objects appearing in the image. In a step 156, for each identified object, the system determines values for a plurality of physical attributes of the respective identified object. In a step 158, the system then synthesizes a vocalized verbal description of the respective identified object, wherein, in a step 160, at least some of the values of said plurality of physical attributes are expressed as non-verbal audio parameters of the synthesized vocalized verbal description. Finally, in a step 162a, the system outputs the synthesized vocalized verbal description to a user through a loudspeaker or an earphone. Alternatively, in step 162b, the system may construct a soundscape representing multiple objects in the scene, and then output the soundscape in a step 164 to the user.

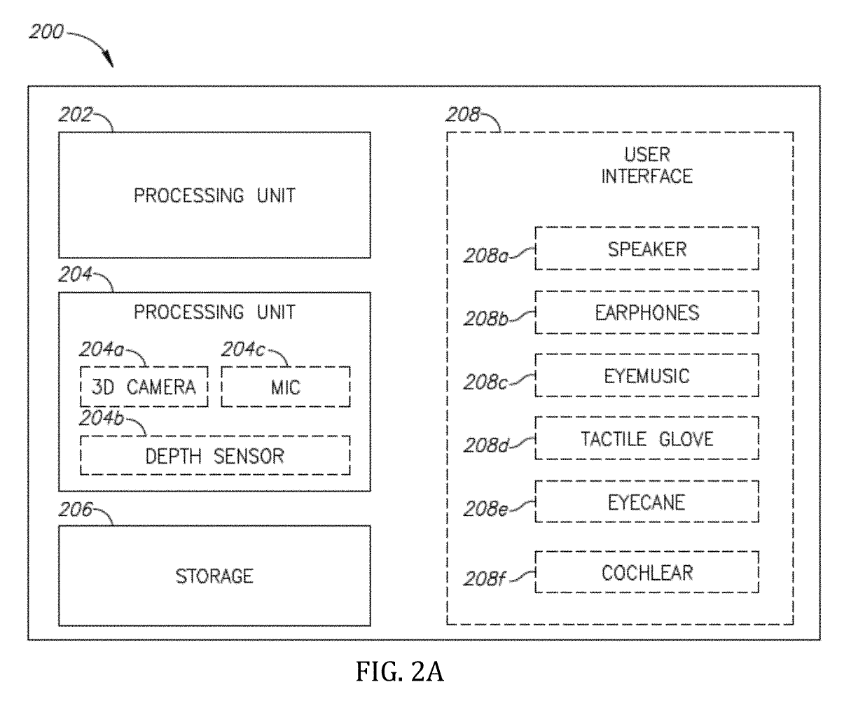

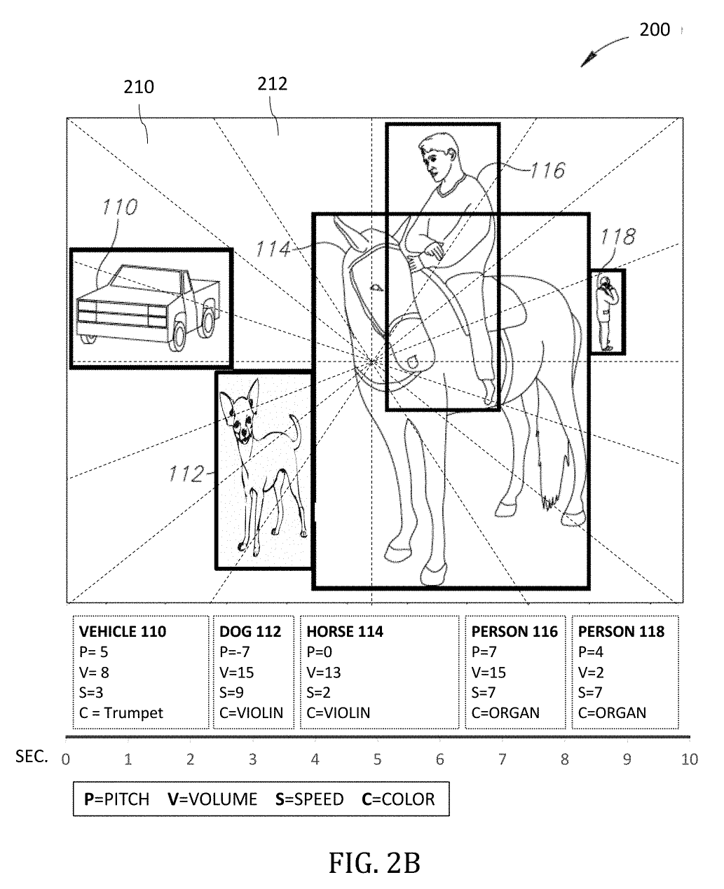

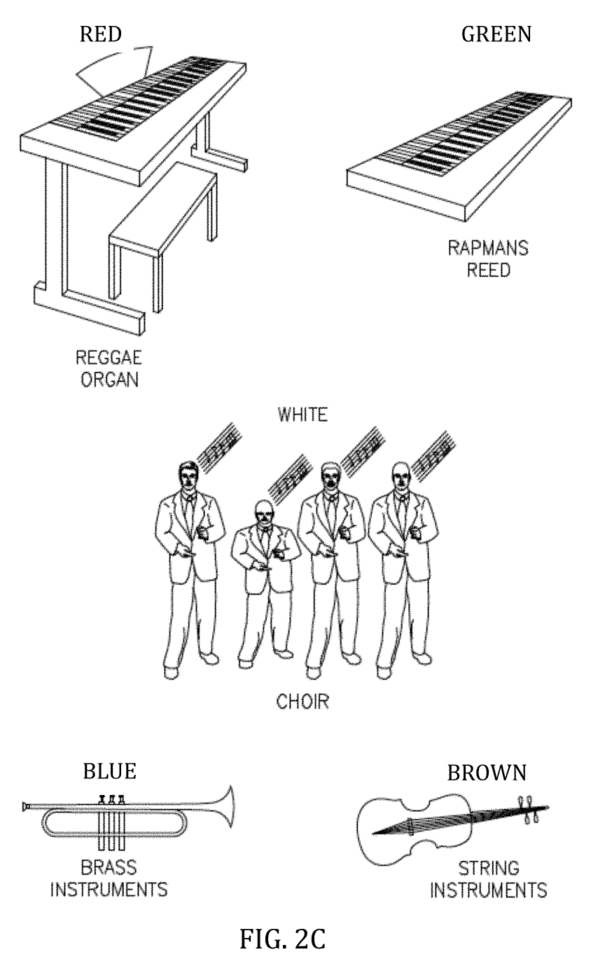

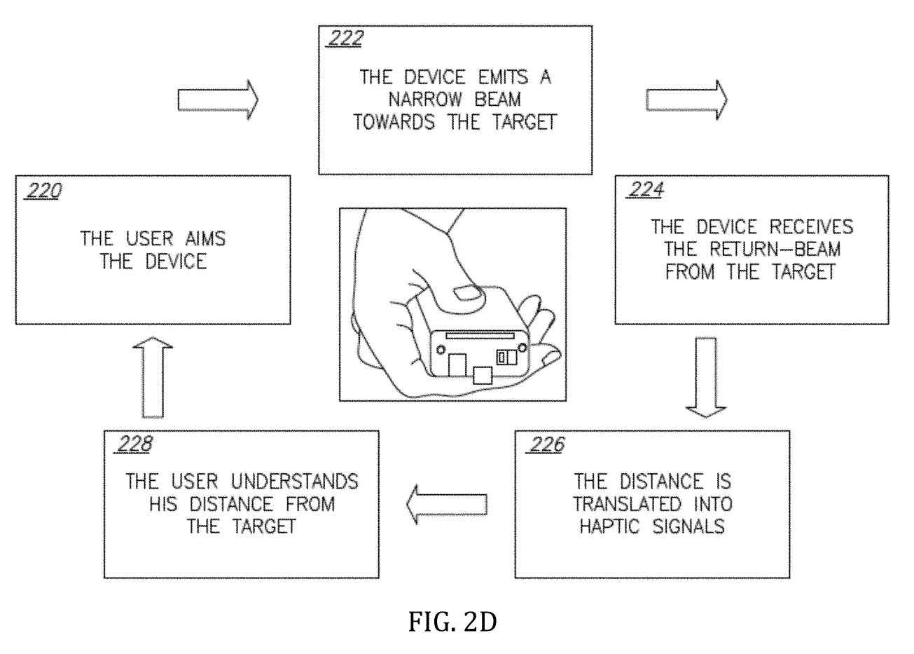

FIG. 2A shows a block diagram of an exemplary embodiment of a system 200 for conveying physical attributes of objects in a scene through non-verbal audio parameters of vocalized verbal descriptions. Processing unit 202 is operatively coupled to sensing unit 204, non-transitory computer readable storage medium 206, and user interface unit 208. Sensing unit 204 may comprise, e.g., a 3D or similar camera 204a, depth sensor 204b, and microphone 204c. Storage medium 206 may comprise software, such as computer readable program instructions for execution by processing unit 202. Storage 206 may also have stored there on a variety of algorithms and applications, including, but not limited to, object recognition algorithms (e.g., YOLO, R-CNN), facial recognition algorithms (e.g., OpenFace, FaceNet), and/or machine learning or deep learning algorithms (e.g., TensorFlow, Caffe). User interface 208 may comprise a variety of user interface devices and implements through which system 200 may output vocalized verbal descriptions and other sensory signals, including loudspeaker 208a, earphones (or headphones or bone-conductors) 208b, EyeMusic device 208c, tactile glove 208d, EyeCane device 208e, and/or a cochlear implant 208f.

In some embodiments, system 200 may comprise, or be used in conjunction with, additional user-interface devices and implements, which may provide a richer experience in conveying additional information, such as exact pixel-by-pixel representation of a scene. For example, in some variations, system 200 may comprise an EyeMusic device 208e, or another similar device having one or more of the features of the device disclosed in the afore-mentioned PCT International Application No. PCT/IB2010/054975, International Filing Date Nov. 3, 2010, published on May 12, 2011 as International Patent Application Publication No. WO 2011/055309. EyeMusic 208c is more fully described in A. Amedi et al., “EyeMusic: Introducing a “visual” colorful experience for the blind using auditory sensory substitution,” Restorative Neurology and Neuroscience, 2013. EyeMusic 208e conveys raw visual input of a scene through non-vocal audio and/or tactile representation, wherein the representation focuses on slice-by-slice position information with respect to objects in the scene. Thus, whereas the present system conveys the identity and spatial position of an object as a whole, EyeMusic 208e can convey the raw shape or outline of an object, as it is positioned and oriented in the scene. For example, EyeMusic 208e may represent the same object differently, depending on whether it is positioned vertically, horizontally, or diagonally. As illustrated in FIG. 2B, EyeMusic 208e does this by slicing an image into a sequence of slices a specified order, for example, slices 210, 212 of scene 200, detecting in each slice a feature distinguishable from the background (which feature may be a part of a larger object contained in the scene), and associating a sound and/or tactile signal indicating the location of such feature in the scene. For example, EyeMusic 208e may sound a piano key (or another musical instrument), upon detection of such feature, wherein the tone of the piano key corresponds to the physical location of the feature (e.g., high tone—high location). Thus, as EyeMusic sweeps through a scene in a specified order, a picture of the shape or outline of the object and its orientation emerges. For example, in the case of a book in a scene, the present system may verbalize the word “book,” and represent the spatial location of the book within the scene, based, e.g., on the location of its geometric center. When used in conjunction with EyeMusic 208e, EyeMusic 208e may further supplement this information with data relating to the actual orientation of the book in its spatial location, e.g., whether it is positioned standing up, lying on its side, or leaning diagonally against another object to the left or to the right. EyeMusic 208e may also convey the exact color of an object, by associating, e.g., the sound of a specified musical instrument with each color, as illustrated in FIG. 2C.

The non-vocal audio signals generated by EyeMusic 208c may be output by system 200 concurrently with a soundscape produced by system 200. Accordingly, with reference to FIGS. 1A, 2A and 2B, the spatial location of vehicle 110 may be represented by system 200 as a vocalized verbal description identifying vehicle 110 as “vehicle,” with the appropriate non-verbal audio parameters denoting the spatial location of vehicle 110 as a whole within scene 100. Concurrently, EyeMusic 208e may represent the outline shape and orientation of vehicle 110 within scene 100, as well as convey its color as “blue” using the sound of a brass instrument. Thus, by combining EyeMusic 208c into system 200, users may be able to receive in one synchronous output a fuller picture accurately representing the identity, location as a whole, outline shape, orientation, and/or color, etc., of objects in the scene. This combination of these top-down (object identities) and bottom-up (the raw visual input, pixel-by-pixel) approaches is expected to benefit the users in gaining new perceptual experience of their surroundings.

In some embodiments, system 200 may comprise, or be used in conjunction with, a tactile user interface, such as tactile glove 208d. In one variation, tactile glove 208d may convey visual information related to the shape of different elements or the topography of the scene concurrently with a soundscape synthesized by system 200, which may convey information on, e.g., the identity, size, and spatial locations of objects in the scene.

With reference to FIG. 2D, in other embodiments, system 200 may comprise, or be used in conjunction with, in a non-limiting example, an infrared-based, ultrasound, and/or laser device for guiding blind and visually impaired persons, such as an EyeCane device 208e, having one or more of the features of the device disclosed in U.S. Patent Application Publication No. 2014/0055229 to Amedi et al. In a step 220, the user aims the EyeCane device 208e at an object. EyeCane 208e emits in a step 222 a narrow infrared beam)(<5° in the direction at which the device is pointed. In a step 224, EyeCane 208e receives a return beam from the target, which modifies the baseline voltage in an electrical circuit of EyeCane 208e, thus translating the distance from the detected object into a DC voltage signal. This DC-voltage signal is translated in real-time in a step 226 into haptic signals, e.g., vibration amplitudes and frequencies, enabling instantaneous feedback to the user in a step 228, such that the closer an object is to the user the stronger and higher the frequency of vibration. In some variations, the haptic signals generated by EyeCane 208e may be output concurrently with a soundscape of system 200, such that, e.g., vibration amplitudes and frequencies will supplement the soundscape to denote distance from objects in a scene.

With reference again to FIG. 2A, in some embodiments, system 200 may comprise, or be used in conjunction with, a cochlear implant 208f. Cochlear Implants are devices that aim at replacing normal cochlear function by directly stimulating the auditory nerve with electric impulses, conveying signal to the acoustic nerve. After the implantation, patients may undergo a rehabilitation program, which mainly trains them in hearing and comprehending speech. However, such rehabilitation programs typically do not focus on other types of important sound characteristics, such as pitch or volume, and moreover, show a huge variability of results among subjects. System 200 may be employed as a training method for cochlear implant patients. In such embodiment, patients may hear the soundscapes synthesized by system 200, and perform different tasks related to them and to their relation to the visual output. This way, participants will not only train on comprehending speech, but also on different sound characteristics, and will do it in a multisensory way, in which the visual image (which can be comprised of objects, texts, faces and/or other objects) can be sometimes displayed and sometimes not, according to the training method.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A system comprising:
at least one hardware processor configured to:
receive a digital image of a scene;
analyze the image to identify one or more objects appearing in the image; and
for each identified object:
(i) determine values for a plurality of physical attributes of the respective identified object,
(ii) synthesize a vocalized verbal description of the respective identified object, wherein at least some of the values of said plurality of physical attributes are expressed by non-verbal audio parameters of the synthesized vocalized verbal description, and
(iii) output said synthesized vocalized verbal description through a loudspeaker or an earphone.

2. The system of claim 1, wherein said plurality of non-verbal audio parameters are selected from the group consisting of: pitch, volume, timbre, speed, voice gender, number of voices used, type of voice used, language, accent, emotion expressed by the voice, echo, and reverberation.

3. The system of claim 1, wherein said plurality of physical attributes are selected from the group consisting of: location in the horizontal dimension, location in the vertical dimension, location in the depth dimension, height, width, size, color, depth, weight, texture, temperature, identity of a human, sex, height of a human, weight of a human, age of a human, nationality of a human, and emotional state or mood of a human.

4. (canceled)

5. The system of claim 1, wherein said object comprises at least one of textual information and symbolic information, and wherein said identification comprises detection of said textual or symbolic information.

6. (canceled)

7. The system of claim 1, wherein said identification comprises retrieving information with respect to said identified object from at least one of a database of the system, an external network resource, a cloud server, and the Internet.

8. The system of claim 1, wherein a plurality of said synthesized vocalized verbal descriptions corresponding to a plurality of objects disposed in different locations about a said image, are combined into a continuous sequence in a specified order, based on the relative locations of said plurality of objects in the image, and wherein said specified order is selected from the group consisting of: left-to-right, right-to-left, top-to-bottom, and bottom-to-top.

9. (canceled)

10. The system of claim 1, wherein a said vocalized verbal description of said respective identified object comprises two or more concurrent vocalized verbal descriptions.

11. The system of claim 1, wherein said at least one hardware processor is further configured to:
slice the image into a plurality of slices;
detect, in a specified order for each slice at a time, the location of at least a portion of the object contained within the slice;
associate a sound or tactile object-dependent signal with the object;
associate a sound or tactile location-dependent signal unique for each slice;
combine in the specified order each object-dependent signal with a respective location-dependent signal for creating a combined object-location signal; and
output the combined object-location signal concurrently with said synthesized vocalized verbal description.

12. The system of claim 1, wherein each of the non-verbal audio parameters is associated with a unique physical attribute, based on user selection.

13. The system of claim 1, wherein at least some of the determined values for said plurality of physical attributes of a said identified object are expressed by haptic signals, and wherein said haptic signals are being output concurrently with said vocalized verbal description of the respective identified object.

14.-18. (canceled)

19. A method comprising using at least one hardware processor for receiving a digital image of a scene;
analyzing the image to identify one or more objects appearing in the image; and
for each identified object:
(i) determining values for a plurality of physical attributes of the respective identified object,
(ii) synthesizing a vocalized verbal description of the respective identified object, wherein at least some of the values of said plurality of physical attributes are expressed by non-verbal audio parameters of the synthesized vocalized verbal description, and
(iii) outputting said synthesized vocalized verbal description through a loudspeaker or an earphone.

20. The method of claim 19, wherein said plurality of non-verbal audio parameters are selected from the group consisting of: pitch, volume, timbre, speed, voice gender, number of voices used, type of voice used, language, accent, emotion expressed by the voice, echo, and reverberation.

21. The method of claim 19, wherein said plurality of physical attributes are selected from the group consisting of: location in the horizontal dimension, location in the vertical dimension, location in the depth dimension, height, width, size, color, depth, weight, texture, and temperature, identity of a human, sex, height of a human, weight of a human, age of a human, nationality of a human, and emotional state or mood of a human.

22. (canceled)

23. The method of claim 19, wherein said object comprises at least one of textual information and symbolic information, and wherein said identification comprises detection of said textual or symbolic information.

24. (canceled)

25. The method of claim 19, wherein said identification comprises retrieving information with respect to said identified object from at least one of a database of the system, an external network resource, a cloud server, and the Internet.

26. The method of claim 19, wherein a plurality of said synthesized vocalized verbal descriptions corresponding to a plurality of objects disposed in different locations about a said image, are combined into a continuous sequence in a specified order, based on the relative locations of said plurality of objects in the image, and wherein said specified order is selected from the group consisting of: left-to-right, right-to-left, top-to-bottom, and bottom-to-top.

27. (canceled)

28. The method of claim 19, wherein a said vocalized verbal description of said respective identified object comprises two or more concurrent vocalized verbal descriptions.

29. The method of claim 19, further comprising the steps of:
slicing the image into a plurality of slices;
detecting, in a specified order for each slice at a time, the location of at least a portion of the object contained within the slice;
associating a sound or tactile object-dependent signal with the object;
associating a sound or tactile location-dependent signal unique for each slice;
combining in the specified order each object-dependent signal with a respective location-dependent signal for creating a combined object-location signal; and
outputting the combined object-location signal concurrently with said synthesized vocalized verbal description.

30. (canceled)

31. The method of claim 19, wherein at least some of the determined values for said plurality of physical attributes of a said identified object are expressed by haptic signals, and wherein said haptic signals are being output concurrently with said vocalized verbal description of the respective identified object.

32.-36. (canceled)

37. A computer program product, the computer program product comprising a non-transitory computer-readable storage medium having program code embodied therewith, the program code executable by at least one hardware processor to:
receive a digital image of a scene;
analyze the image to identify one or more objects appearing in the image; and
for each identified object:
(i) determine values for a plurality of physical attributes of the respective identified object,
(ii) synthesize a vocalized verbal description of the respective identified object, wherein at least some of the values of said plurality of physical attributes are expressed by non-verbal audio parameters of the synthesized vocalized verbal description, and
(iii) output said synthesized vocalized verbal description through a loudspeaker or an earphone.

38.-53. (canceled)
